(19) United States
(12) Patent Application Publication
(10) Pub. No.: US 2019/0046142 A1
(43) Pub. Date: Feb. 14, 2019

(54) MAMMOGRAPHY DEVICE

(71) Applicant: Yasmin FAKHARI TEHRANI, Vaughan (CA)

(72) Inventor: Yasmin FAKHARI TEHRANI, Vaughan (CA)

(21) Appl. No.: 16/100,862

(22) Filed: Aug. 10, 2018

Related U.S. Application Data
(60) Provisional application No. 62/544,361, filed on Aug. 11, 2017.

(51) Int. Cl. A61B 6/00; A61B 6/04

(52) U.S. Cl. CPC A61B 6/502; A61B 6/0414; A61B 6/0435

(57) ABSTRACT

The present invention is a mammography device that holds a woman's breast in a comfortable manner for mammogram procedure comprising of a radiation application unit and a pair of right and left cups are installed in the compartment to insert patient's breast for mammogram procedure. Each cup provides an adjustable system to be able to be adjusted to various body sizes. The adjustable system includes a tubular mesh to flew compressed air and a plurality of sensors to capture images. The device is wirelessly controllable by a remote control.

### FIELD OF THE INVENTION

The present invention relates in general to mammography devices and in particular to a mammography device capable to be adjusted to various body sizes and control the strength of force to be applied to the breast of the patient.

### BACKGROUND OF THE INVENTION

Cancer affects thousands of people worldwide per year and breast cancer is a type of cancer that is often not routinely checked for until it is in its late stages, because of the painful and embarrassing processes used to detect it. One of the current processes involve intense pressing of a patient's breasts in between two panels, another involves repeated pressure being placed upon a patient's breasts with a hand-held machine.

The U.S. Preventive Services Task Force recommends women, age 50-74, have a mammogram every other year but many women in the world skip mammograms because they had a painful experience. Women report that did not go back for their breast monitoring because their last mammogram was so excruciating.

In a mammogram, a breast is compressed and flattened to avoid motion blurring, to enhance the image and to reduce the dose of radiation. With current mammography systems, technicians depend on the machine's measurement of force to get a sense of how much they need to squeeze a woman's breasts to get a good image. Current machines in use in examination settings around the world cause, or can cause, discomfort and pain to the user, because in the design of the machines, pressure is needed to be applied to an individual's breasts to take a correct image.

Studies on mammography pain and discomfort vary, but according to a 2008 review, up to 35 percent of women report pain from the procedure. Although many women deal with it, some may delay or avoid recommended mammograms altogether. One study found that among women who didn't return for their second annual mammogram, 46 percent cited a painful first screening as the reason. Many women should take pain relief pills after the procedure.

### SUMMARY OF THE INVENTION

The present invention is a mammography device that holds the woman's breast in a comfortable manner and prevents the breast being squeezed between two very solid, and usually cold, plates. The mammography device of the present invention can be used in all patients with all body size, whether individuals with abnormally larger breasts to individuals with abnormally smaller breasts without causing any discomfort. The present invention further eliminates the radiation to be pervaded to the other part of the body making it safer.

The mammogram device of the present invention comprises of a moveable compartment connected to a moveable arm of a base section through a rotary shaft. The compartment moves in all directions in front of a patient's body for adjusting the compartment on the body of the user. The compartment has a radiation application unit (radiation generator). A pair of right and left cup are installed in the compartment to insert patients' breast for mammogram procedure. Each cup provides a plurality of image capturing sensors and have an adjustable system to be able to be adjusted to various body sizes.

Radiation application compartment applies radiation to the breast of the patient from different directions through the movement of the radiation source, and a radiation image capturing sensors detect radiation image generated through the application of radiation by the radiation application section with respect to each of the image capturing directions.

The cups of the mammography device of the present invention have a unique design. Each cup comprise of an inner layer attached from its circumference edge to the cups. The inner layer is usually of material with conforming nature so as to purposefully cling and grip to the body for example, thermoplastic polyurethane polymers, spandex or latex may be used for this application. Each of intrinsically sticky fibers holds the breast in place for a period to capture the images.

Each cup has an adjustable system to be able to be adjusted to various body sizes. The inner layer comprises of conforming mesh of thin tubes, which will be filled with compressed air through ports into the tubes. The air pressure is controlled by the compression system to control the amount of the pressure required for each patient tissue. The system assist the Conforming Mesh in fitting snugly around a patient's breasts creating a personalized fit.

The mammogram device of the present invention may include an elastic member that expands or contracts to secure the patient in front of the device and prevent any movement during the procedure while radiation image capturing is performed, whereby the body posture of the subject may be stabilized.

The mammogram machine retrofitted with a pressure control system to control compression pressure. A pressure detector for controlling the amount of the pressure provided on each breast of the patient according to the inputs of the device. The device have predetermined data for patients with various breast sizes and amount of compression pressure, eliminating some of the variation inherent in the system.

In a mammography device for capturing a breast image, radiation is applied from a radiation source unit to the breasts of the patient. The unique design of the present invention prevents radiation emitted from the radiation source unit from being applied to the other parts of the body of the patient. A plurality of sensors are provided on to the inner layer of the cups on various locations and intended to send “readings” of the captured images from different directions.

A handheld remote controller by the operator wirelessly communicates with various controlled members of the mammography device. The operator can remotely control the movement of the compartment to initiate up and down, left and right, back and forth movement or rotation, remotely activate and control the air pressure system of the cups which is inputted in the database of the computer system.

It is an objective of the present invention to provide a device to encourage more people to be examined and monitored.

It is another objective of the present invention to make mammography more comfortable and less of a humiliating experience, which may cause persons to get more timely and more frequent examinations and possibly prevent death.

It is another object of the present invention to minimize the pain many women feel during the mammogram process without any loss of image quality.

It is another object of the present invention to encourage more women to go through the procedure routinely by providing a painless device.

### BRIEF DESCRIPTION OF THE DRAWINGS

Embodiments herein will hereinafter be described in conjunction with the appended drawings provided to illustrate and not to limit the scope of the claims, wherein like designations denote like elements, and in which:

FIG. 1 is a perspective side view of the mammography device of the present invention;

FIG. 2 is a top view illustrating the operation of the imaging system of the present invention;

FIG. 3 is a top view illustrating the adjustable system of the present invention;

FIG. 4A is a top view of the inner layer of the right cup according to the present invention;

FIG. 4B is a top view of the inner layer of the left cup according to the present invention;

FIG. 5 is a diagram showing the remote controlling system of the present invention, and

FIG. 6 is a diagram illustrating image capturing procedure according to the present invention.

### DETAILED DESCRIPTION OF PREFERRED EMBODIMENTS

The figures are not intended to be exhaustive or to limit the present invention to the precise form disclosed. It should be understood that the invention can be practiced with modification and alteration, and that the disclosed technology be limited only by the claims and equivalents thereof.

The device disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

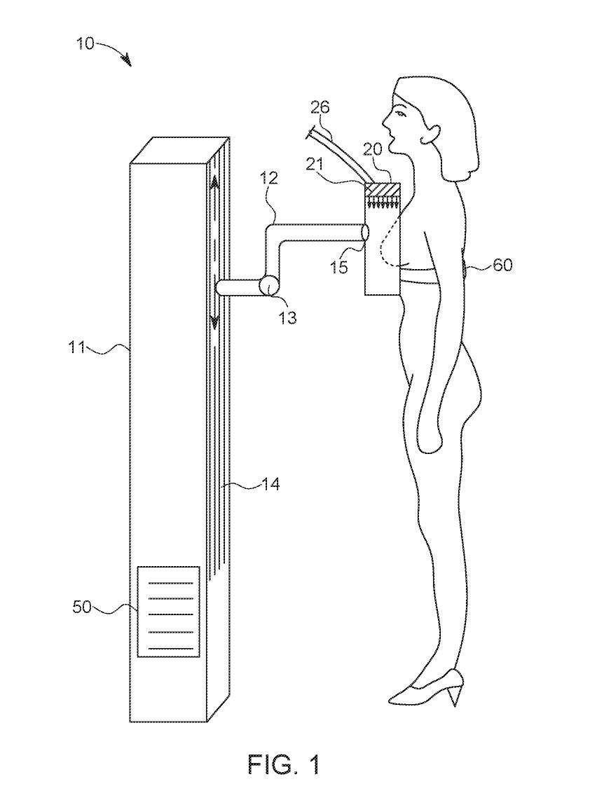

As shown in FIG. 1 the mammography device of the present invention 10 includes a base 11, an L shape arm 12 connected to the base 11 from its proximal end by a first rotary shaft 13. The L shape arm is moveable in an up-down direction on a railing 14 provided on the base. The arm 12 is moveable in all directions (X-Y-Z) with respect to the base 11.

A compartment 20 is rotatably attached to the distal end of the arm 12 by a second rotary shaft 15. The compartment 20 is preferably a rectangular housing including a radiation application unit and breast receiving units for the mammography procedure.

The mammography device of the present invention 10 provides a computer system 50 to wirelessly communicate with various controlled members of the device 10.

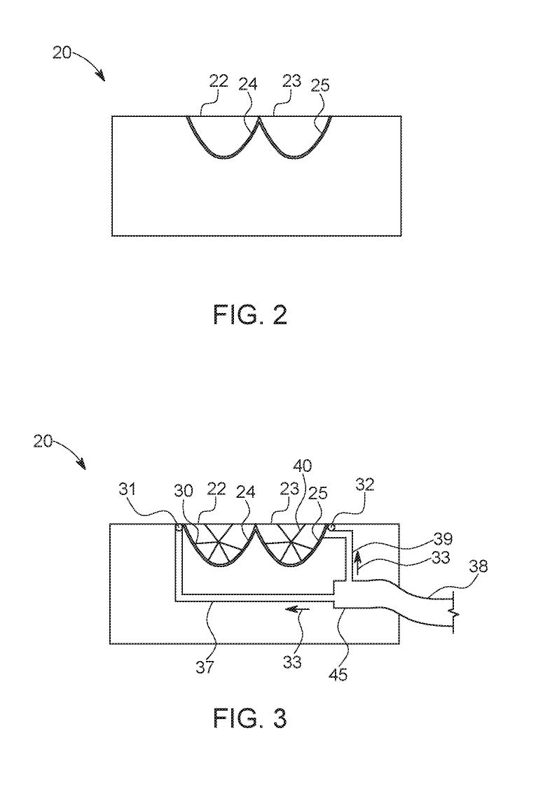

According to FIG. 2 the compartment 20 comprises of a radiation application unit 21 that emits radiation to the breast receiving cups. The radiation application unit 21 is mounted on the location that can emit radiation on both breast or on each breast at a time and can be controlled by the operator. A hose 26 connects the radiation application unit 21 to the radiation source.

The breast receiving units 22 and 23 are two perforation mounted on the front side of the compartment 20, which comprises of two hemisphere shape cup, a right-cup 22 and a left-cup 23 to insert patients breast. The cups 22 and 23 have an inner layer 24 and 25 in same hemisphere shape and size of the cups 22 and 23 of a hypoallergenic soft material. The inner layer 24 and 25 is usually of material with conforming nature to purposefully cling and grip to the body for example; thermoplastic polyurethane polymers, spandex or latex may be used for this application. The material combine the elasticity of a polymer or a cloth with metal or optical fibers to facilitate the device's operation. Each of intrinsically sticky fibers holds the breast in place for a period to capture the images. The inner layer 24 and 25 allow the cups to fit snugly to a person without being tight.

The inner layer 24 and 25 have means to attach to the circumference edge of the cups 22 and 23 and are changeable. The inner layer 24 and 25 should be produced in, from a range of considerably small to considerably large and comfortably accommodate thinner patients, and larger patients. Each of the cups have an adjustable system to be able to be adjusted to various sizes.

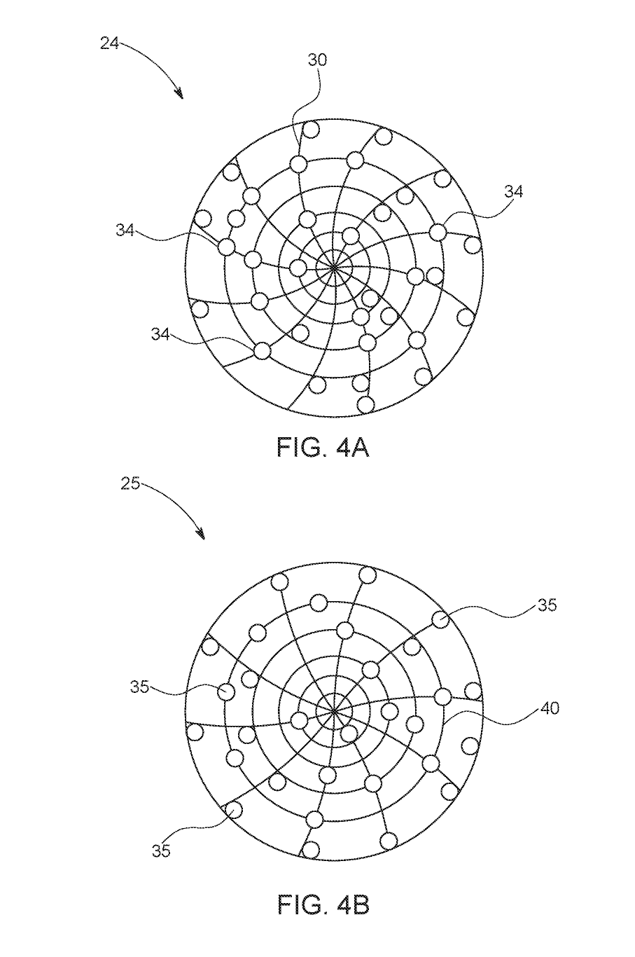

According to FIGS. 3, 4A, 4B and 6 the adjustable system of the cups 22 and 23 comprises of a conforming mesh 30 and 40 of thin tubes on the inner layer 24 and 25 which will be filled with compressed air. Two ports 31 and 32 for applying air into the tubes are provided for each cup 22 and 23. Said ports 31 and 32 are installed on the circumference of each cup 22 and 23. The airflow 33 is blown through an air pressure system 45 mounted inside the compartment 20 into the tubes 30 and 40 to assist the conforming Mesh in fitting snugly around a patient's breasts creating a personalized fit. Two thin tubes 37 and 39 direct the compressed air from the air pressure system into the mesh tubes 30 and 40. The air pressure system 45 controls the amount of the pressure required for each patient. This pressure amount is stored in the computer system. The arrows show the direction of the flow of the compressed air into the hoses 37 and 39 to direct the airflow into the mesh hoses 30 and 40 on each cup 24 and 25. The air pressure system 45 is connected to an air pressure unit through a hose 38.

A plurality of Sensors 34 and 35 are provided on to the inner layer of the cups 24 and 25. The Sensors 34 and 35 correspond to various locations and intended to send “readings” along Mesh Lines 30 and 40. In the breast image, capturing it is important to perform stereoscopic image capturing from different directions. There are many Sensors 34 and 35 on the inner layers 24 and 25 in various sizes. Sensors 34 and 35 are connected to one another with Mesh Lines 30 and 40. The Mesh Lines 30 and 40 are connected to the Rim located on the circumference of both of the cups 22 and 23. The Sensors 34 and 35 cover the Conforming Mesh's surface area and along with the Mesh Lines 30 and 40, facilitate complete readings of a patient's breast and sends and receives the signals of the breast tissue through wireless data transfer methods to a computer operated by a medical technician or doctor. It is sometimes desirable to capture images from a specific spot. This can be accomplished with the present invention.

The Sensors 34 and 35 will be able to read breast tissue using the best method available, whether electromagnetic fields, light or auditory waves, nanotechnology, etc., and will create and send a 2 or 3 D image of the patient's breasts, to a computer, allowing a doctor to view and interpret clinical findings. Each of the Sensors 34 and 35 have a specific identifiable placement in the device's design. This may enable ease in maintenance, or reading of findings by a doctor or technician. Methods for the radiation image signal readings are well known in the prior art.

According to FIG. 1 again the device 10 provides straps 60 with hook and loop fasteners to hold the patient stay in a stable position in front of the device 10. The straps 60 continuous and extend across the entirely of the wearers back and fasten with a hook and loop fastener.

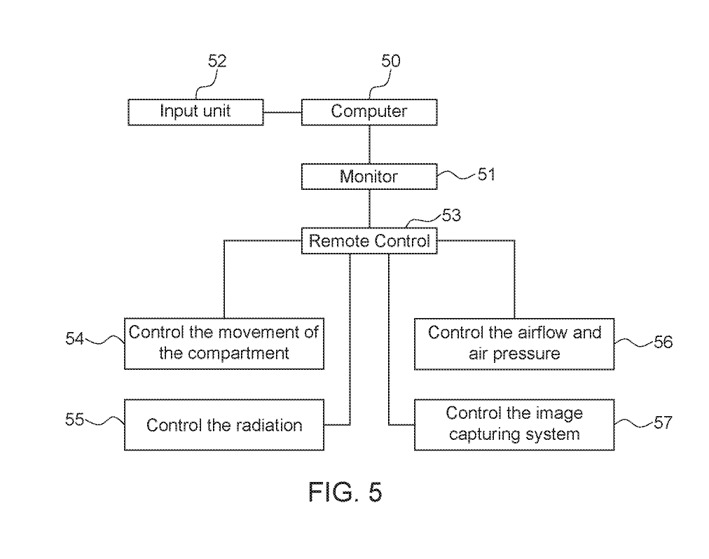

According to FIG. 5 the mammogram device 10 comprises of a computer 50 connected to the mammogram device 10. The computer 50 comprises a monitor 51 and an input unit 52 connected to the computer 50 with a remote control system 53. The operator may input the information to the input unit. The movement of the compartment 54 is controlled by the computer system. The emission timing of radiation from the radiation source 55, the airflow control system 56 further will be controlled by the computer system.

The handheld remote controller 53 wirelessly communicates with various controlled members of the mammography device 10. In the system the operator can remotely control the movement of the compartment 20 to initiate up and down, left and right, back and forth movement or rotation, remotely control the pressure on patients body which is inputted in the database of the computer system, activate and control the air flow system of the cups 56.

The computer 50 further includes a radiation image capturing section 57 which captures, and stores image signals detected by the sensors 34 and 35.

An operation of the breast image capturing system of the present embodiment will now be described with reference to the present invention 10. First, a breast of the patient is placed into the image capturing cups 22 and 23. The cups 22 and 23 align to receive the breasts. The operator will then fasten the straps 60 to keep the patient in place during the procedure. The air start to flow 33 from the air pressure system 45 into the mesh tubes 30 and 40 on inner layer of each cup 24 and 25 so that the breast is compressed at a predetermined pressure by the cups. The air compressed in mesh tubes 30 and 40 in combination with the sticky material of the inner layer of the cups 24 and 25, will allow the wearer to achieve a great fit, facilitating a more comfortable mammography.

Then, when the breasts of the patient is pressed by the inner layer of the cups 24 and 25 an instruction to start image capturing 57 is given by the instructor through the remote control input 53 to the radiation application unit 55 to apply radiation and to the sensors 34 and 35 to capture images. All the procedure is controlled by the computer system 50 of the device.

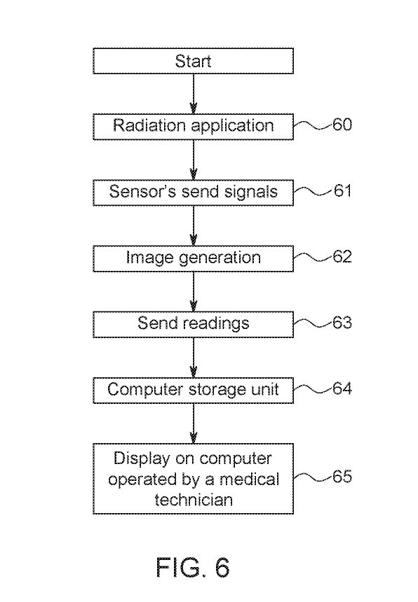

FIG. 6 is a diagram illustrating the procedure of image generating of the present invention. The radiation applies from radiation application unit in the compartment 60. By moving the compartment, the radiation applies to the breasts from all directions. The sensors on the breasts of the patient receive signals from the breast and can obtains information from a target on the breast 61.

The image generation unit generates a 2D or 3D images in accordance with the signals and displays the generated image in the display unit 63. The image generation unit may obtain subject information by performing a process based on an image reconstruction algorithm on the reception signal 64. The amount of radiation to be applied and the examination area is based on the inputs information of the device. The images generated will be stored in storage unit 64 and display on display unit to be discussed by the medical technician or physician 65.

The foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

With respect to the above description, it is to be realized that the optimum relationships for the parts of the invention in regard to size, shape, form, materials, function and manner of operation, assembly and use are deemed readily apparent and obvious to those skilled in the art, and all equivalent relationships to those illustrated in the drawings and described in the specification are intended to be encompassed by the present invention.

What is claimed is:

1. A mammography device comprising of a radiation application unit moveably attached to an arm, said arm has a distal end and a proximal end; wherein said distal end of said arm is moveably attached to a base and said proximal end of said arm is further moveably attached to said radiation application unit, said radiation application unit further comprising a pair of breast receiving cups, wherein each said cups have an inner layer attached from its circumference edge to said cups, each said cup has an adjusting system and a plurality of image capturing sensors and a radiation image detector for capturing and detecting a radiation image generated through said application of radiation by said radiation application unit.

2. The mammography device of claim 1, wherein said radiation application unit is respectfully moving in an up and down, left and right and rotation direction in front of a user and applies radiation through a radiation source mounted in said radiation application unit to a breast of said user from different image capturing directions.

3. The mammography device of claim 1, wherein said radiation application unit further comprising a radiation application source.

4. The mammography device of claim 1, wherein said radiation application unit further comprising an air pressure source.

5. The mammography device of claim 1, wherein said adjusting system comprising of thin tubes constructed in a conforming mesh shape on said inner layer of said cups, wherein said tubes are filled with compressed air through said air pressure source to assist said conforming Mesh in fitting snugly around said user's breasts creating a personalized fit.

6. The mammography device of claim 1, wherein said inner layer of said cups are made of intrinsically sticky materials so as to purposefully cling and grip to the body of said user and hold the breast of said user in place for a period to capture the images and constructed in a mesh form tubes.

7. The mammography device of claim 1, wherein said inner layer of said cups are attached from its circumference edge to said cups by attaching means or attaching mechanism;
Whereby said inner layer can be changed according to the body sizes and hygiene proposes.

8. The mammography device of claim 1, wherein said mesh tubes are connected to said air pressure system by a pair of thin hoses which direct the air flow into said inner layer of said cups, said mesh tubes of said cups are filled with compressed air through a pair of ports mounted on circumference of said cups.

9. The mammography device of claim 1, wherein a computer system is provided to wirelessly control the various members of said device.

10. The mammography device of claim 1, wherein said air pressure system controls the amount of the pressure required for each patient based on predetermined data inputs for each patient with various breast sizes.

11. The mammography device of claim 1, wherein said predetermined data inputs for each patient with various breast sizes are stored in said computer system.

12. The mammography device of claim 1, wherein said image capturing sensors and detectors detect a radiation image generated through the application of radiation by said radiation application unit with respect to each image capturing direction and intended to send “readings” of said captured images from different directions.

13. The mammography device of claim 1, wherein said device include a strap with fastening means to secure the patient in front of said device and prevent any movement during the procedure while radiation image capturing is performed.

14. A method of imaging a patient's breast onto a mammogram comprising:
positioning a patient in front of a mammogram device and aligning said device so that the breast of said patient is positioned into said breast receiving area of said device;
fastening said straps to secure the patient in front of said device and avoid any movement during the procedure;
injecting compressed air into said tubes of said inner layer of said cups such that said inner layer expands in said cup and the breast of patient is compressed;
transmitting radiation through radiation source to the breast, and
capturing images by said image capturing sensors and sending reading.
